(19) United States
(12) Patent Application Publication
(10) Pub. No.: US 2019/0168272 A1
(43) Pub. Date: Jun. 6, 2019

(54) Wax Warmer Liner

(71) Applicant: Rebecca Ann Treadway Fancher, West Monroe, LA (US)

(72) Inventor: Rebecca Ann Treadway Fancher, West Monroe, LA (US)

(21) Appl. No.: 16/231,093

(22) Filed: Dec. 21, 2018

(51) Int. Cl. B08B 17/04; A61L 9/03

(52) U.S. Cl. CPC B08B 17/04; A61L 9/03

(57) ABSTRACT

A method of containing and removing melted wax or therapeutic oils (usually scented) while protecting the integrity of the receiving portion, or reservoir, of a warming mechanism, known typically as a wax warmer or diffuser. Such container (hereafter referred to as “liner”) being placed in direct contact with the base of the reservoir of the warming mechanism with wax or oils being placed inside the liner, rather than being placed directly into the reservoir of the warming mechanism itself. Such liner would simplify the process of removing the used melted or solid wax or oils from the reservoir of the wax warmer, providing a convenient method of disposing of used wax or oils and reducing or eliminating the messiness and potential dangers of traditional methods of removal. The liner also could be used as a storage container or for reusing removed wax or oils.

### BACKGROUND OF THE INVENTION

### Field of the Invention

The present disclosure relates to a containment device (liner) to be placed into the receiving portion of a warming mechanism used to melt or warm wax or scented wax or therapeutic oils, usually for the purpose of releasing aroma into the ambient atmosphere of an environment, protecting the receiving portion, or reservoir, of the warming mechanism, and presenting a simple, safe and effective removal of the melted or solid wax or oil product and acting as a method to dispose or store and reuse removed wax or oils.

### Description of the Background of the Invention

Melting or warming wax and oils has become a popular way of improving the ambient atmosphere of an environment, such as home, workplace or office. As the wax or oils warm, they release aroma or therapeutic properties into the air, creating a usually pleasant scent or therapeutic response. Warmers are generally electric, using low-wattage bulbs to gently heat the receiving portion of the warmer that holds the wax or oils. Other types of warmers employ the use of votive-style candles, allowing the open flame to warm the receiving portion of the warmer that holds the wax or oils, and others use electric heating devices.

Warming is preferred as a method of reaping the benefits of scent or aromatherapy, as opposed to burning, such as with an open flame in a candle, which changes the molecular properties of the wax or oils, and may render them ineffective or, in some cases, may even release toxins as well as pose other dangers, such as creating a fire hazard.

Melted wax has always posed a problem with candles or wax warmers. Candles drip, but some of the wax is burned up and dissipates into the air as it burns. Blowing out the candle can cause melted wax to spew onto surrounding surfaces, which can cause damage by the molten wax embedding into fabric or porous surfaces, and creating another problem with cleanup.

Wax warmers, while providing a containment portion (hereafter referred to as “reservoir”) for the wax itself, pose similar problems with the removal and cleanup of molten, melted, softened or solidified wax. There are at least three types of warmers: a wall plug-in warmer, which is usually small enough to fit directly into an electrical outlet; a larger warmer used to put on a countertop, desk or table, with a length of electrical cord used to plug into an electrical outlet; and warmers that actually use a small, votive-style candle to warm the tray of the warmer with open flame. Regardless of the type of warmer, wax removal and disposal is always a problem, and those who use wax are often discouraged by the inconvenience and difficulty of removing used wax. This often leads to the abandonment of the functional and therapeutic use of warmed waxes or oils.

There are several ways to remove used wax from the tray of a warming mechanism. 1) The molten wax can be poured out. If the warmer is a small plug-in version, the entire mechanism sometimes must be removed from the wall electrical outlet, as often the smaller versions do not have a detachable tray. The pulling motion to remove the warmer from the electrical outlet often causes the wax to spill out and onto the surrounding surfaces or onto the background wall in the removal process. The melted wax must quickly be poured out of the reservoir before the wax becomes solid. The reservoir must then be immediately wiped out, or cleaned, to prevent wax buildup which is not only messy but can blend the remaining scent of the residual wax with the subsequent wax or oils that may next be added to the reservoir. With the larger warmers, the reservoirs usually are removable, but in the transport of the reservoir to the receptacle, trash or garbage container, the wax is prone to sloshing out or spilling and immediately solidifies onto the surrounding surfaces, floors, fabrics, clothing, or with whatever it comes in contact. Again, the residual wax must be wiped out before it solidifies, or must be scraped out if it has solidified, to clean the reservoir and prevent the scent or oils from combining with any new wax or oils that may be added. 2) Wax that is not removed immediately while it is still molten, melted or warmed, will solidify quickly and will result in becoming embedded in the tray of the warmer until it is warmed again, and removed while melted, or chipped away until it is removed. Wax that is solid can be removed by taking a sharp object and loosening the edges to pry the wax out, or to chip the wax until it is removed, or the reservoir may be inverted onto a surface to be tapped to try to loosen the solid wax. Either way is not preferred, as one involves potentially dangerous activity involving sharp objects, and the other involves a possible problem with permanently damaging or breaking the tray or reservoir of the warmer. 3) Molten, melted or warmed wax may be removed from the reservoir of a warmer by placing absorbent material directly into the wax and soaking up the liquid. This method does eliminate the sloshing but if not done properly, can result in wax dripping from the material onto the surrounding surfaces, and causing the problems listed in examples 1 and 2.

Of the three removal methods listed, none is convenient, and none presents a completely effective means of cleaning the reservoir of a wax warmer. The present invention provides a means of containing the wax or oils placed in the reservoir of a warmer and acts as a removal device without compromising the position of the warmer or warmer trays, thus eliminating the pulling, jerking, sloshing, spilling, wiping or chipping that occurs with the other means of wax removal while preserving the integrity of the reservoir itself, eliminating the wax buildup that inevitably occurs with repeated use of wax warmers, and allows the user to change waxes frequently and a means for storing usable wax for later use.

### BRIEF DESCRIPTION OF THE DRAWINGS

FIG. 1 is a perspective view of a round wax-warming mechanism with the present invention included.

FIG. 2 depicts the invention of the round version of the liner holding an unused block of wax

FIG. 3 depicts the invention holding melted wax, and indicating the sides and bottom of the round version of the liner.

FIG. 4 is a perspective view of a square wax-warming mechanism with the present invention included.

FIG. 5 depicts the invention of the square version of the liner holding an unused block of wax.

FIG. 6 depicts the invention holding melted wax, and indicating the sides and bottom of the square version of the liner.

FIG. 7 is a view of the bottom portion of the round version of the liner.

FIG. 8 is a side view of the round version of the liner.

FIG. 9 is the view of the bottom portion of the square version of the liner.

FIG. 10 is a side view of the square version of the liner.

FIG. 11 is a perspective view of a round wax warming mechanism incorporating a concave reservoir with present invention included.

FIG. 12 depicts the invention of a concave version of the liner shown from a bottomward perspective.

FIG. 13 is a perspective view of a round wax warmer mechanism incorporating a convex reservoir with present invention included.

FIG. 14 depicts the invention of a convex version of the liner shown from an topward perspective.

### LIST OF ELEMENTS

1) Round liner
2) Wax block
3) Round warming mechanism
4) Melted wax
5) Perpendicular side wall of round liner
6) Bottom of round liner
7) Square liner
8) Block of wax
9) Square mechanism
10) Melted wax
11) Bottom of square liner
12) Perpendicular side wall of square liner
13) Flange associated with the liner of the concave liner
14) Concave liner
15) Convex liner
16) Flange associated with the liner of the convex liner

### DETAILED DESCRIPTION OF THE DRAWINGS

The present invention, a liner for scented wax warmers, provides a solution to the problem of wax removal from the receiving portion, or reservoir, of a wax-warming mechanism, the purpose of warming wax being for aromatic or therapeutic purposes. The invention is a containment device, or liner, that receives wax, scented wax or therapeutic oils and is positioned into the receiving portion of a warming mechanism, forming a barrier between the wax and the reservoir. This allows for simple and safe removal of the wax, either molten or cooled, to a solid element.

FIG. 1 is a perspective view of a round warming mechanism (3) showing the placement of the round containment device, or liner (1). The round liner (1) is seated in position within the reservoir (13) of the wax warming mechanism. The block of wax (2) is shown perspectively placed inside the round liner (1). FIG. 2 is the round liner (1) in its entirety, round, or circular in shape, and containing the block of wax (2). FIG. 3 illustrates the round liner (1) containing melted wax (4). The bottom portion of the roundly designed liner (6) is indicated, and the wall or side of the liner is shown in a perpendicular fashion (5).

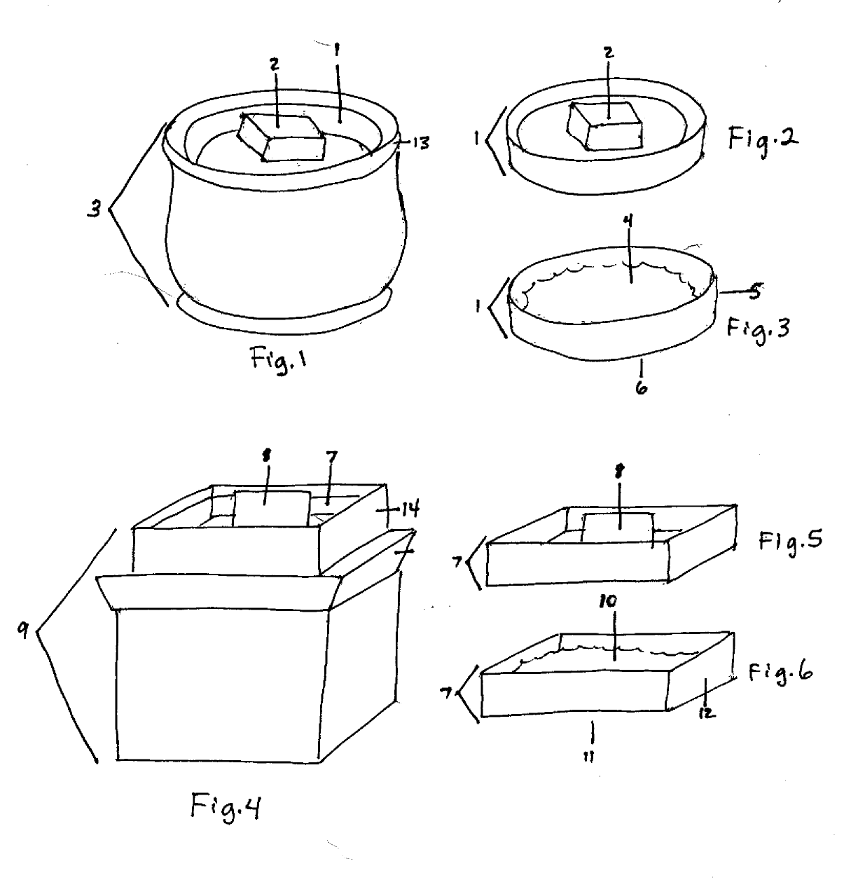

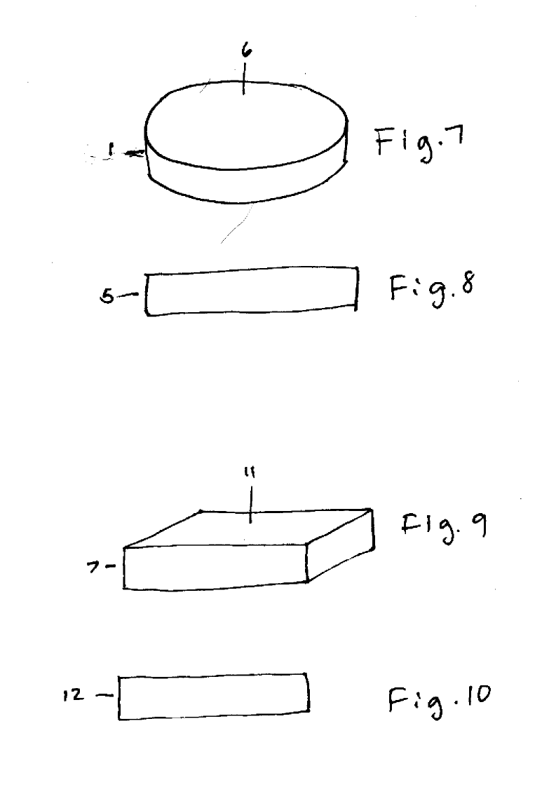

A squarely designed warming mechanism (9) is shown in FIG. 4. The square liner (7) is indicated as a square design, fitting inside the square receiving portion, or reservoir (14) of the warming mechanism (3). FIG. 5 is a view of the square liner (7) holding the block of wax (8), positioned inside the square liner (7). In FIG. 6, the block of wax has melted (10) inside the square liner (7), and the bottom portion (11) and sides or walls (12) of the square liner (7) are shown in perpendicular fashion.

FIG. 7 is a view of the bottom portion (6) of the round liner (1) in an inverted state. FIG. 8 indicates the side or wall (5) of the round liner (1).

FIG. 9 is a view of the bottom portion (11) of the square liner (7) in an inverted state. FIG. 10 indicates the sides or walls (12) of the square liner (7).

FIG. 11 depicts a round wax warming mechanism (3) incorporating a concave reservoir, with the concave liner (14) and liner flange (13).

FIG. 12 is a closer view of the concave liner (14) and the flange (13).

FIG. 13 depicts a round wax warming mechanism (3) incorporating a convex reservoir, with the convex liner (15) and liner flange (16).

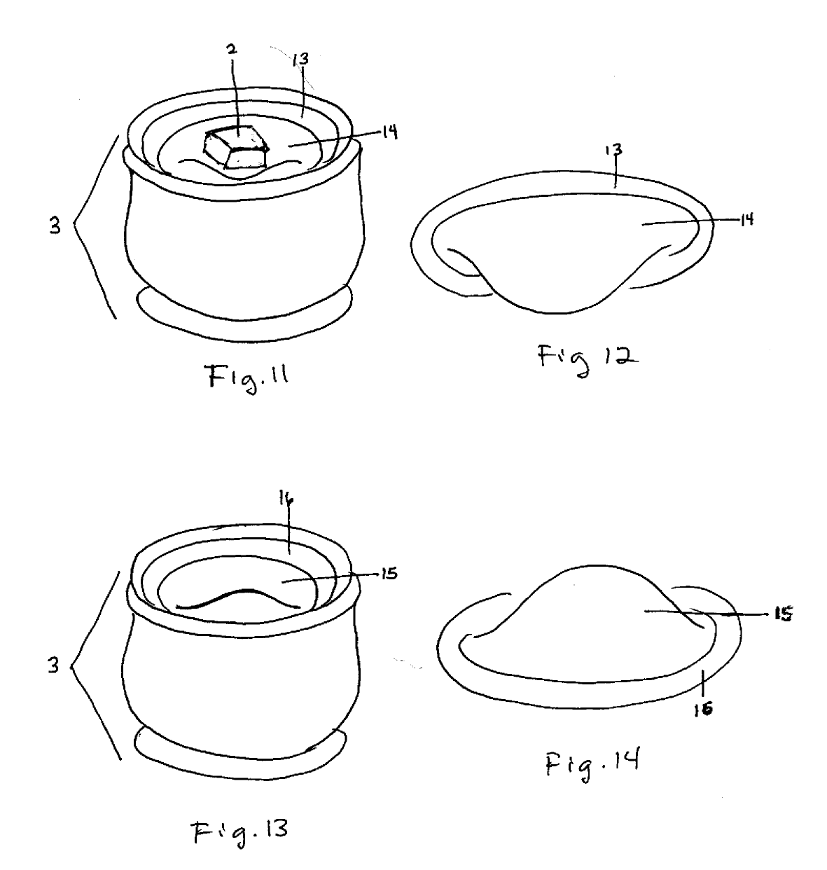

FIG. 14 is a closer view of the convex liner (15) and the flange (16).

### DESCRIPTION OF RELATED ART

The findings of related art particularly relate to the wax warming mechanism itself, or the wax scented cubes, rather than to the reservoir liner, which is the current invention.

U.S. Pat. No. 10,086,102 B2, applicant S.C. Johnson & Son, Inc. relates to a Wax Warmer, which is an electrical warming mechanism containing a reservoir for holding a solid wax cube, which will become molten upon warming. This patent references traditional warmers and candles and their drawbacks in favor of their invention while there is no mention of the present invention, a liner for the reservoir.

Likewise, U.S. Pat. No. 10,118,466 B1, applicant Meri Horn, relates to Scented Pasty Wax Delivery System and Method and Composition references including “removable cartridges with or without sponges that can soak up the melting scented, pasty wax mixture to be dispursed throughout the space so that fragrances pass into the spaces.” This invention clearly indicates a bag type device used to dispense the pasty wax mixture by squeezing the mixture into a receiving mechanism, and is unrelated to the present wax warmer liner invention.

Other patents document inventions relating to the wax itself. U.S. Pat. No. 10,010,638 B2, S.C. Johnson & Son, Inc. refers to a Wax Melt With Filler and contains claims and information pertaining to the wax cube product itself, with no mention of the present invention. U.S. Pat. No. D764,274 S, The Proctor & Gamble Company refers to a Package for Wax Articles which pertains to the device that holds wax for the purpose of storage and sale and not designed for use with a wax warming mechanism. There is no mention of the present invention.

The need for and usefulness of the present invention is realized through the adequate lack of related art.

### SUMMARY OF THE INVENTION

The present invention, the liner for wax warmers, when placed in the reservoir of a wax warmer (a housing for a heat source, often a light bulb, to provide low temperature heat directly to a reservoir above the heat source, which holds scented wax or therapeutic oils to spread ambient aroma throughout an atmosphere, such as an office or a home), provides a simple, safe and effective means of containing and removing, disposing or storing scented or aromatic wax or therapeutic oils. When the warmer is turned off, or the heat source is removed, the wax quickly becomes solid. If the wax or oils are still useful, with scent or therapeutic aroma still remaining, the wax or oils can be reused simply by reintroducing the heat source to the wax or oils stored in the liner. If the user desires to change the wax or oil, the liner may be removed and the contents stored for another use at a later time, and a new liner placed in the reservoir with a different wax or oil. This allows the user diversity, while not wasting the still useable portion of wax or oil for the purpose of changing a scent or therapeutic value, by discarding it while the properties are still effective. Wax that has cooled in a liner may be loosened and removed easily by popping out, and can be saved for a later use, while the liner can be reused for another wax, if undamaged. Liners also can be disposable and a different liner used each time for fresh wax or oil.

The liners are presented in a variety of sizes, to be used universally in wax warmers of various brands, and not be specific to any particular brand. The preferred material of the liner would be plastic, or polymeric material, such as PET or LDPE plastics for optimal results pertaining to heat transfer and conduction to allow the wax to melt through the material, without melting the material itself. A paper product would not be preferred material, as the paper may absorb the wax material or therapeutic oils and retain the properties, preventing the maximum benefits from being realized. A product made with metal or metallic materials would enhance and increase the heat effect from the warming mechanism in the wax warmer and would overheat the wax or oils, causing them to use their effectiveness at a greater rate of speed, which would shorten the use time, or the excessive heat may cause combustion.

The base of the typical liner consists of a bottom surface, preferably to lay flush with the base of the wax warmer reservoir, to maximize the heat from the heat source and allow the heat to transfer through the liner to melt the wax. The liner may have sides that rise up from a flat surface base to form a wall around the circumference or edges of the base to contain the melted wax and prevent spilling. The sides of the wall may be perpendicular, at a 90 degree angle, or may be angled outward at the top to produce extra area for holding more wax. Liners made with angled sides may be stacked together easily for more effective packaging. Still other types of wax warming mechanisms may have a reservoir that is convex or concave in design. A liner to accommodate the convex or concave reservoir of certain wax warmers would be curved, either upward or downward and may incorporate a flange design to stabilize the curved liner from freely moving and contain the molten wax once the wax block is melted.

While the liner may be produced in different sizes, it also may be produced in different shapes, ideally, but not limited to round, square, oval, wavy, curved, fluted, convex or concave. Most wax warming mechanisms, which are typically round or square, either wall mounts or countertop or tabletop versions, will have a flat surface diameter of the wax reservoir of between 1½″ to 3½″, depending on the style. A universal size would fall within this range but is not limited to these dimensions.

The height of the wall and the size of the base can vary, as well as the shape or design of the wall. The height of the wall of the liner will be adequate enough to hold a typical square of a wax melt and allow a little extra space to allow for additional or bigger pieces of wax to be used, if desired. The height should be at least ½″ or taller but not limited to these measurements. The target height would be to be enough to contain the wax, without spilling over, but preferably not taller than the sides of the wax warmer reservoir itself, as to be both aesthetically pleasing as well as practical. A convex or concave version would have an approximate ½″ depth at the peak of the curve, the sides sloping upward or downward toward a flange, which serves as a stabilizing feature of the liner.

The material of the liner ideally would be polymeric material, such as PET or LDPE, and can be clear or colored in appearance. Other materials may be used but are not preferred.

What is claimed to be new and desired to be protected is set forth in appended claims:

1. A liner, acting as a containment device, for the purpose of holding wax, scented wax or therapeutic oils within the receiving portion, or reservoir, of a wax-warming mechanism.
a) a liner, of claim 1, made from, but not limited to, polymeric material.
b) a liner, of claim 1, to be made from material that is reusable.
c) a liner, of claim 1, to be made from material that is disposable.

2. A liner used to contain molten wax during the warming process.

3. A liner purposed for easy removal of warmed or cooled wax or oils from the reservoir of a wax-warming mechanism.

4. A liner, used for storage of the cooled, unused portion of wax, removed from the reservoir of a wax-warming mechanism.

5. A liner, which may be emptied and reused.

6. A liner, used as a barrier to protect the receiving portion, or reservoir, of a wax-warming device from wax buildup and to maintain a new and clean appearance.

7. A liner to prevent scent mixing by maintaining the cleanliness of the receiving portion, or reservoir, of a wax-warming device, and preventing wax buildup that can taint subsequent wax usage of a different scent.
